(19) United States
(12) Patent Application Publication
(10) Pub. No.: US 2011/0084231 A1
(43) Pub. Date: Apr. 14, 2011

(54) Method and process of producing short chain fatty acids from waste stream containing phenolic lignin model compounds by controlled photocatalytic oxidation with titanium dioxide nanocatalyst in the presence of ultraviolet radiation

(76) Inventors: Jerald A. D. Lalman, Windsor (CA); Srimanta Ray, Windsor (CA)

(21) Appl. No.: 12/923,728

(22) Filed: Oct. 5, 2010

Related U.S. Application Data
(60) Provisional application No. 61/272,567, filed on Oct. 6, 2009.

(51) Int. Cl. C07C 51/16; C07C 51/255; C07C 51/34; C07C 57/15; C07C 57/145; C07C 55/10; C07C 53/08; C07C 53/02; B82Y 30/00

(52) U.S. Cl. 252/182.12; 562/537; 562/538; 562/512; 977/773; 977/811; 977/902; 977/835

(57) ABSTRACT

A method of producing short chain carbon compounds from effluents that are rich in lignin-model compounds. The method is characterized by controlled photocatalytic degradation of lignin model compounds so as to produce short chain carbon compounds. The present invention provides converting recalcitrant and toxic organic compounds into chemicals which are of commercial value.

### RELATED APPLICATIONS

This application claims the benefit of 35 USC § 119(e) to U.S. Provisional Patent Application Ser. No. 61/272567, filed Oct. 6, 2009.

### FIELD OF THE INVENTION

The invention pertains to a method of producing short chain carbon compounds, particularly fatty acids, from effluents that are rich in phenolic lignin model compounds.

### BACKGROUND OF THE INVENTION

Wastewater from the paper and pulp industry contains lignin and phenolic lignin degradation products from pulp and paper processes [1]. In the pulping process, the hemicelluloses portion of the plant material is utilized while lignin is produced as a by-product [2]. Lignin is a highly cross-linked, complex, carbon compound composed of three different phenylpropanoid blocks [3-4], associated with the hemicellulosic core of plant tissue and occurs naturally in cell wall of plants [3-5]. High molecular weight and non-hydrolyzable cross-linked structures make the lignin-rich effluent poorly biodegradable in conventional biological wastewater treatment processes [6-9]. Thus, treatment and disposal of lignin-rich, wastewater from paper and pulp industry is a challenge from an environmental perspective. Due to the recalcitrant nature, the most common disposal method of the lignin-rich effluent is to dry and incinerate the dried residue. The disposal method of drying followed by incineration of lignin residues is neither environmentally friendly nor energy efficient or economically attractive [1]. Several investigators have examined different alternatives to effectively utilize lignin, a naturally-occurring cheap carbon source, to produce some recoverable intermediates of commercial importance [6, 10-12].

Aerobic and anaerobic bacteria do not degrade lignin with success but fungal communities have been reported to successfully degrade lignin [2]. Ascomycetes, such as Xylaria sp., Libertella sp., Hypoxylon sp., collectively known as “white-rot fungi” are able to degrade lignin and lignin residues. However, white-rot basidomycetes, P. chrytosporium is reported to be the most efficient species in mineralizing lignin [13,14]. Degradation of lignin is catalyzed by fungal oxidative enzyme and proceed via aromatic ring cleavage and progressive depolymerization. Fungal degradation of lignin is slow and thereby its applicability in treating effluent of pulp and paper industry is severely limited [13-15].

Recent attention has been focused towards oxidative degradation of lignin [13]. Strong oxidant, such as the hydroxyl radical (.OH), has been reported to be particularly successful in degrading lignin into carbon dioxide [8]. In aqueous medium, *OH radical can be produced by different techniques either in singular or in combination [16]. Generation of .OH radical on the surface of photo-irradiated titanium dioxide (TiO₂) in the presence of singlet oxygen or hydrogen peroxide is one such technique, which has received significant research attention in recent years [17]. Formation of the .OH radical on the surface of TiO₂ particle originates from the semiconductor band gap of titanium dioxide. On irradiating TiO₂ with electromagnetic radiation having energy higher than its intrinsic band gap, a pair of conduction band (CB) electron (e⁻) and valence band (VB) hole (h⁺) is generated. The charge carriers, i.e. electrons in conduction band or hole in valence band either recombine with the bulk of the material or migrate to the particle surface. In aqueous medium, this electron-hole pair initiates an oxidation—reduction reaction at the particle surface to produce hydroxyl radicals (.OH) which subsequently cause the degradation of organic molecules [18-19]. Enhanced formation of .OH radicals is also expected in presence hydrogen peroxide or singlet oxygen is aqueous medium. Consequently, incremental increases in the degradation rate of organic compounds have been reported upon the addition of hydrogen peroxide or singlet oxygen in a photocatalytic system [20-21].

Photo-oxidation of lignin model compounds in aqueous medium through the hydroxyl radical initiated pathway by ultraviolet light in presence of titanium dioxide catalyst have been reported in literature. Photocatalysis of lignin model compounds in aqueous medium proceeds by ring opening and subsequent degradation of aromatic moieties into simple aliphatic carboxylic acid intermediates which eventually degrade into carbon dioxide (CO₂) and water [2,8,12, 22-25]. Pigmentary TiO₂ particles in the micrometer range thereby lacks photocatalytic activity mainly due to recombination of charge carriers en-route to the catalyst surface. Augmenting the TiO₂ photocatalytic efficiency is expected to be dependent on the specific surface area of the catalyst or reducing the diffusion path. Studies have shown improved photocatalytic efficiency for TiO₂ particles in the nanometer range [26-27].

### SUMMARY OF THE INVENTION

The present invention provides a process wherein strict control of the variables of the TiO₂ photocatalysis of lignin model compounds results in the production of commercially value-added byproducts, such as, short chain carboxylic acids and their derivatives.

A further purpose of this invention is to provide a process to treat effluents which are rich in lignin model compounds and to reduce the hazard of recalcitrant waste stream on the environment.

Yet a further purpose of the present invention is to utilize photocatalytic degradation in a process of controlled degradation of lignin model compounds. Herein, the proper control of process variables allows of the production of commercially value end products from an effluent rich in lignin-model compounds.

Thus, the method is characterized by controlled photocatalytic oxidation of lignin model compounds by titanium dioxide nanocatalysts in the presence of ultraviolet radiation to produce short chain fatty acids, including but not limited to, formic acid, acetic acid, succinic acid, fumaric acid, maleic acid and their derivatives. The present invention aims at converting recalcitrant and toxic phenolic compounds into chemicals which are of commercial value.

Accordingly, in one aspect the invention provides a process of controllably photocatalytically degrading lignin residues in aqueous solution, the process comprising treating the lignin residues in the presence of a titanium dioxide nanocatalyst with ultraviolet radiation.

Preferably, the lignin residue is present in an aqueous waste stream, particularly a recalcitrant efficient stream rich in lignin model compounds, selected from the group consisting of phenol, syringol and guaiacol.

By the terms “controllably” and “controlled” is meant that the process conditions are so selected as to limit, when desired, the total decomposition of the lignin residues to CO₂ as to stop the decomposition at the short chain fatty acid stage.

Such conditions of pH, UV radiation wavelength, temperature, duration, TiO₂ nanocatalyst particle size, oxidising agent and nature and concentration of the lignin residue model compound, to suitably effectively produce the fatty acids, can be determined by the skilled person.

Preferably, the process as hereinabove defined comprises controlled photocatalytic cracking with ultraviolet radiation of lignin residues in the presence of the titanium dioxide nanocatalyst and an oxidising agent, particularly an oxidising agent selected from the group consisting of dissolved oxygen, hydrogen peroxide and ozone.

Although not so limited, preferably the term “short chain” fatty acid defines C₂ to C₁₀ linear or branched chain alkyl or alkenyl group. Preferred short chain carboxylic acids are selected from the group consisting of formic acid, acetic acid, succinic acid, maleic acid, fumaric acid and their derivatives.

Preferably, the titanium dioxide has a nanoparticle size to provide an activation energy of between 5-50 kilojoule per mol of the lignin residue and of at least 10 nm.

Preferably, the process as hereinabove defined is wherein said aqueous solution has a pH of less than 7, more preferably from 1 to 3.

Preferably, the titanium dioxide nanocatalyst is immobilized on a support material.

The process as hereinabove defined may comprise a batch process, or a continuous or semi-continuous mode.

The applicant has found that the concentration and type of short chain carboxylic acid obtained varied with the phenolic substrate, oxidant concentration, exposure time and reaction temperature. The rate of photocatalysis on the TiO₂ surface increased with decreasing TiO₂ particle size and an increase in TiO₂ specific surface area, to an optimum particle size diameter of 10 nm, beyond which there was no further incremental increase in the photocatalytic degradation rate. Further, the phenolic substrates degraded faster with increased oxidant concentration and lower substrate concentration, while the reaction rate of the photocatalysis of the lignin residues in the aqueous solution increased with a decrease in the UV wave length and increase in the irradiance of the UV light.

In addition, it has also been found that the photocatalytic degradation rate of the lignin residues increased with an increase in TiO₂ nanoparticle loading until a threshold value, beyond which increase in TiO₂ nanoparticle loading did not cause an increase in the reaction rate.

As indicated, hereinabove, the optimum TiO₂ nanoparticle size, the photocatalysis of lignin model compounds, including but not limited to phenol, syringol and guaiacol, followed an apparent first order degradation kinetics and had an activation energy between 5-50 kilojoule (kJ) per mol of the reactant. An increase in reaction temperature within a range, including but not limited to, 10-60 ° C., was favourable towards faster photocatalytic degradation of the lignin residues by TiO₂ and UV in the presence of oxidizing agents. The photocatalytic degradation rate of the lignin residues proceeded via hydroxyl substitution in the aromatic ring, followed by oxidative cleavage of the ring to form dicarboxylic acid. Thereafter, subsequent degradation produced simple aliphatic carboxylic acids and ultimately into carbon dioxide (CO₂) and water.

It is further observed that the formation of short chain carboxylic acids from controlled photocatalysis of lignin residues was favorable under acidic pH conditions, within a range, including but not limited to, pH 1-3. We also observed that the use of TiO₂ nanocatalyst immobilized on support material facilitated easy removal of the nanocatalyst after the reaction was completed and allowed of scaling-up the process from batch to semi-continuous mode.

The process of the invention as hereinabove described allows of the production of short chain carboxylic acids, including but not limited to, succinic acid, which is a high-value chemical in the “bio-based economy”. Succinic acid is a precursor for many industrially important chemicals in food, chemical and pharmaceutical industries, including but not limited to, 1,4-butanediol, γ-butyrolactone, tetrahydofuran and methylpyrrolidone. Similarly, the process of the invention provides for the production of maleic acids, which can hydrogenated to 1,4-butandiol, a precursor for biodegradable polymers, and acetic acid which is a feedstock chemical for microbial fuel cell.

In a further aspect, the invention provides an aqueous solution comprising a short-chain fatty acid when produced by a process as hereinabove described.

In yet a further aspect, the invention provides a short chain fatty acid obtained by isolating said fatty acid from the aqueous solution as hereinabove defined by an effective suitable method selected from distillation, crystallization, evaporation and chromatography.

### BRIEF DESCRIPTION OF THE DRAWINGS

In order that the invention can be better understood, preferred embodiments will now be described, by way of example only, wherein

FIG. 1 is a schematic diagram of a photocatalytic reactor of use in the □ractice of the invention;

FIG. 2 is a chromatogram identifying short chain carboxylic acids obtained from the controlled degradation of lignin model compounds in a photocatalytic reactions, according to the invention; and

FIGS. 3A and 3B show profiles of the formation of short chain carboxylic acids from the degradation of lignin model compounds in photocatalytic reaction, according to the invention.

### DETAILED DESCRIPTION OF PREFERRED EMBODIMENTS

### (i) General Experimental Apparatus and Procedures

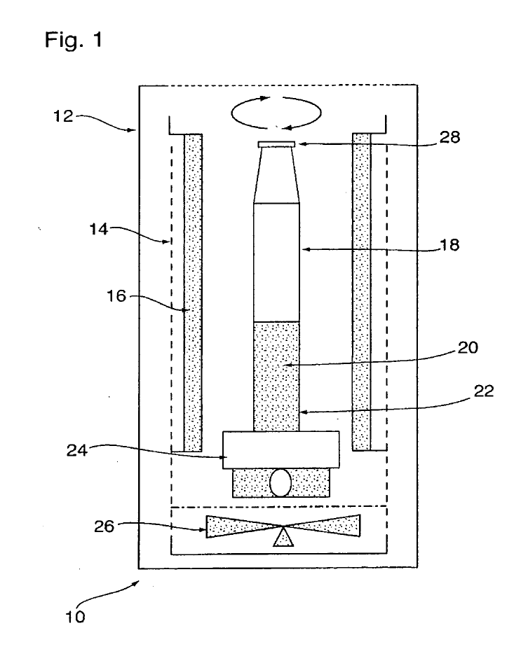

With reference to FIG. 1, which shows generally as 10, a photocatalytic reactor having a temperature-controlled chamber 12, embracing a UV chamber 14 containing a monochromatic UV lamp 16 providing UV radiation at 300 nm on a quartz reaction tube 18 containing aqueous reaction liquid 20. Tube 18 holds a magnet 22 and rests on a magnetic stirrer plate 24 above a circulation fan 26 and is crimped with a Teflon® seal 28.

Degradation of the substrate was monitored using a high performance liquid chromatograph (Dionex Ultimate™ 3000, Sunnyvale, Calif.) which was equipped with a UV-visible photodiode array detector at a wavelength specific to the compound under examination. Degradation products of photocatalysis were identified using a GC-MS (Varian™, Saturn™ 2000, Palo Alto, Calif.) configured with a DB-5MS 0.25 mm (ID)×30 m (length), 0.25 μm (film thickness) column and DBFFAP 0.25 mm (ID)×30 m (length), 0.25 μm (film thickness) column under different setup (P. J. Cobert, St. Louis, Mo.) by comparison against a pure compounds or against National Institute of Standards and Technology (NIST) library spectrum.

### (ii) EXAMPLES

In one non-limiting example, an aqueous mixture of lignin residue model compounds, including phenol, syringol and guaiacol, was exposed to ultraviolet (UV) light, of wavelength, of 300 nanometers (nm) and irradiance, of 9 milliwatt per square meter (mW/m²), in the presence of TiO₂ nanocatalysts in aqueous solution containing strong oxidizing agents, dissolved oxygen, hydrogen peroxide and ozone, which favoured the formation of hydroxyl radical, at elevated temperature of about 50° C. using photoreactor 10 shown in FIG. 1. After exposing the reaction mixture containing lignin model compounds for a sufficient period of time in the aforesaid controlled conditions, the reaction mixture was analyzed by high performance liquid chromatography (HPLC). The results indicated the formation of variety of short chain carboxylic acids, including formic acid, acetic acid, succinic acid, fumaric acid and maleic acid.

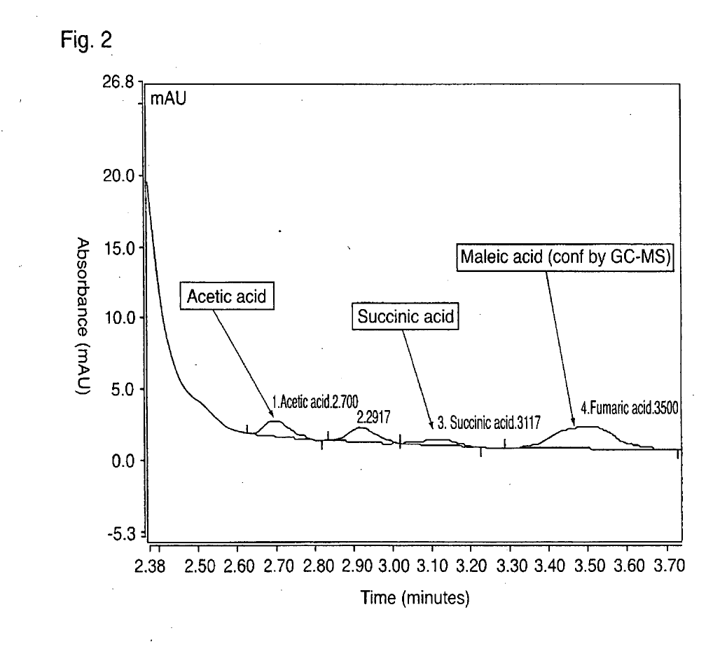

In one such sample experiment, 40 milligram/liter (mg/l) phenol solution was exposed to monochromatic UV radiation of 300 nm wavelength in the presence of 10 nm TiO₂ particles of concentration 1 gram per liter in an aqueous solution containing 31 mg/l dissolved oxygen at a reaction temperature of 50° C. for 60 to 90 minutes. The analysis of the liquid extracts from the reaction mixture by a high performance liquid chromatograph (HPLC) identified the formation of different short chain carboxylic acids, as for example is illustrated in FIG. 2. In particular, analysis shows the formation of at least formic acid, acetic acid, succinic acid and fumaric acid or maleic acid on comparison with standards made from pure compounds. The concentration and type of short chain carboxylic acids varied with the phenolic substrate, oxidant concentration, exposure time and reaction temperature. Faster degradation of the phenolic substrates was observed with increased oxidant concentration and lower substrate concentration. A threshold TiO₂ concentration and TiO₂ nanocatalyst size was recorded with increasing degradation rates.

In further studies, three different TiO₂ particles (Alfa Aesar, Ward Hill, Mass.) with diameters in the range from 5 to 32 nm were used to photocatalytically degrade lignin model compounds (Sigma Aldrich, Oakville, Ontario, Canada). These photocatalysts had identical physical and chemical properties and including crystal structure confirmed by X-ray diffraction. They varied only in particle size and surface area. Photocatalytic experiments were performed in a photocatalytic reactor (25 mm ID×250 mm length), fabricated using GE-214™ clear fused quartz silica (Technical Glass Products Inc., Painesville, Ohio). Sealed reactors containing the model compound in aqueous solution and TiO₂ photocatalyst were placed in a modified Rayonet™ RPR-100 UV photocatalytic reactor 10 (Southern New England Ultraviolet Co., Connecticut) of the type shown in FIG. 1. This custom built reactor 10 was equipped with sixteen phosphor-coated low-pressure mercury lamps 16 on the outer perimeter and a centrally located rotating inner carousel. Six fused quartz reaction tubes 18 were placed on the inner rotating carousel. The lamps 16 (300 nm monochromatic UV light) had an average irradiance of 9 mW/cm² as measured using a calibrated UV-X radiometer. To minimize variation in irradiance among the UV lamps 16, control experiments were performed to optimize the rotational speed of the inner carousel. Water used in all experiments was of ultrapure quality with 18.0 MOhm resistivity drawn from a Milli-Q (Bamstead, Iowa) water purification unit. Over the duration of each experiment, a fixed amount of aqueous solution was withdrawn at specific time intervals and stored in capped aluminum foil wrapped tubes for further analysis.

### (iii) RESULTS

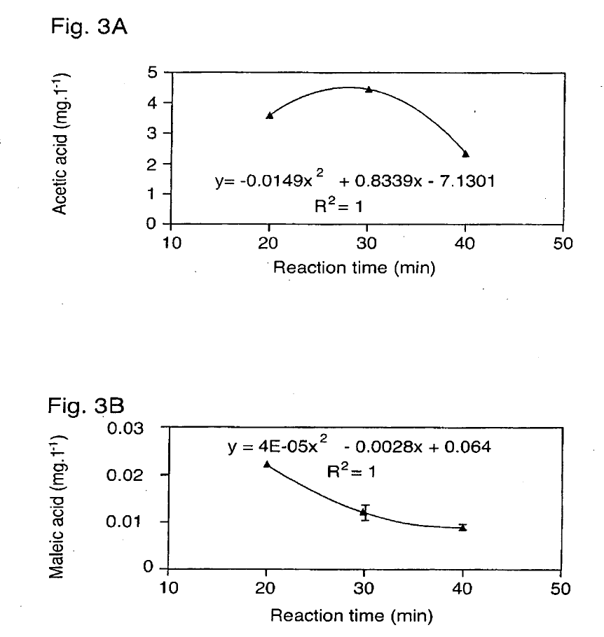

The results showed that controlled photocatalysis of lignin model compounds in aqueous medium proceeded by cleavage of phenyl-propanoid blocks, ring opening and subsequent degradation of aromatic moieties into simple aliphatic carboxylic acid intermediates, which eventually degrade into carbon dioxide (CO₂). Hydroquinone and catechol were identified as the major intermediates from oxidation of the aromatic ring of phenol. Significant amounts of succinic acid, maleic acid, acetic acid were recorded from photocatalysis of phenol using titanium dioxide nanocatalyst irradiated with 300 nm ultraviolet radiation under acidic condition in presence of oxygen at 50° C. in 45 mins, as shown in FIG. 2. Furthermore, as shown best in FIG. 3A and 3B, the formation profile of maleic acid and acetic acid from photocatalytic cracking of phenol (lignin model compound), showed that maleic acid formation precedes acetic acid formation (FIG. 3). Due to non-selectivity of photocatalytic oxidation, short chain carboxylic acids are subsequently degraded into CO₂ and water.

Although this disclosure has describes and illustrates certain preferred embodiments of the invention, it is to be understood that the invention is not restricted to these particular embodiments. Rather, the invention includes all embodiments which are functional or mechanical equivalence of the specific embodiments and features that have been described and illustrated. For a detailed description of the invention, reference may be had to the appended claims.

### REFERENCES

The following publications describe various process and apparatus as related to aspects of the invention heretofore described, and the disclosure of which are hereby incorporated herein by reference.
1. Alder, E. (1977). Lignin Chemistry—Past, present and future. Wood Sci. Tech., 11, 169-218.
2. Font, R., Esparanza, M., Garcia, A. N. (2003). Toxic by-products from the combustion of Kraft lignin. Chemosphere, 52, 1047-1058.
3. Lazalunga, O., Beitti, M. (2000). Invited review: Photo- and radiation chemical induced degradation of lignin model compounds. J. Photoch. Photobio. B., 56, 85-108.
4. Lewis, N. G., Yamamoto, E (1990). Lignin: occurrence, biogenesis and biodegradation. Ann. Rev. Plant Phys., 41, 455-496.
5. http://www.css.cornell.edu/compost/calc/lignin.html
6. Kwon, J. Y., Chung, P. J., Lim, I. H. (2004). Removal of residual COD in biologically treated paper-mill effluent and degradation of lignin using non-thermal plasma unit. J. Environ. Sci. Heal. A., A39, 7, 1853-1865.
7. Pelegrini, R., Reyes, J., Duraan, N., Zamora, P. P., DeAndrade, A. R. (2000). Photoelectro-chemical degradation of lignin. J. Appl. Electrochem., 30, 953-958.
8. Kobayakawa, K., Sato, Y., Nakamura, S., Fujishima, A. (1989). Photodecomposition of Kraft lignin catalyzed by titanium dioxide. B. Chem. Soc. Jpn., 62, 3433-3436.
9. Field, J. A. (2002). Limits of anaerobic biodegradation. Water Sci. Technol., 45(10), 9-18.
10. Anjaneyule, Y., Sreedharachary, N., Suman Raj, D. S. (2005). Decolorization of industrial effluents—available methods and emerging technologies—a review. Rev. Environ. Sci. Biotechnol., 4, 245-273.
11. Suzuki, H., Cao, J., Jin, F., Kishita, A., Enomoto, H. (2006). Wet oxidation of lignin model compounds and acetic acid production. J. Mat. Sci., 4, 1591-1597.
12. Ksibi, M., Amor, S. B., Cherif, S., Elaloui, E., Houas, A., Elaloui, M. (2003). Photodegradation of lignin from black liquor using a UV/TiO2 system. J. Photoch. Photochem. A., 154, 211-218.
13. Kirk, T. K. Farrell., R. L. (1987). Enzymatic “combustion”: the microbial degradation of lignin. Annu.. Rev. Microbiol., 41, 465-505.
14. Tuomela, M., Vikman, M., Hatakka, A., Itavaara, M. (2000). Biodegradation of lignin in a compost environment: a review. Bioresource Technol., 72, 169-183.
15. Evans, W. C., Fuchs, G.; (1988). Anaerobic degradation of aromatic compounds. Ann Rev. Microbiol., 42, 289-317.
16. Gogate, P. R., Pandit, A. B. (2004). A review of imperative technologies for wastewater treatment I: oxidation technologies at ambient conditions. Adv. Environ. Res., 8, 501-551.
17. Herrmann. J. M. (2005). Heterogeneous photocatalysis: state of the art and present applications. Top. Catal.,. 34(1-4), 49-65.
18. Linsebigler, A. L., Lu, G., Yates Jr. J. T. (1995). Photocatalysis on TiO2 surfaces: Principles, mechanisms and selected results. Chem. Rev., 95, 735-758.
19. httl://eprints.cdlr.strath.ac.uk/453/01/Mills_Detoxification.pdf
20. Malato, S., Blanco, J., Richter, C., Braun, B., Maldonado, M. I. (1998). Enhancement of the rate of solar photocatalytic mineralization of organic pollutants by inorganic oxidizing species. Appl. Catal. B-Environ., 17(4), 347-356.
21. Bacsa, R. R., Kiwi, J. (1998). Effect of rutile phase on the photocatalytic properties of nanocrystalline titania during the degradation of p-coumaric acid. App. Catal. B-Environ., 16(1), 19-29.
22. Machado, A. E. H., de Miranda, J. A., de Freitas, R. F., Duarte, E. T. F. M., Ferreira, L. F., Albuquerque, Y. D. T., Ruggiero, R., Sattler, C., de Oliveira, L. (2003). Destruction of the organic matter present in effluent from a cellulose and paper industry using photocatalysis. J. Photoch. Photobio. A., 155(1-3), 231-241.
23. Tanaka, K., Calanag, R. C. R., Hisanaga, T.; (1999). Photocatlyzed degradation of lignin on TiO₂. J. Mol. Catal. A-Chem., 138, 287-294.
24. Machado, A. E. H., Furuyama, A. M., Falone, S. Z., Ruggiero, R., Perez, D. D. S., Castellan, A. (2000). Photocatalytic degradation of lignin and lignin models, using titanium dioxide: the role of the hydroxyl radical. Chemosphere. 40, 115-124.
25. Chang, C. N., Ma, Y. S., Fang, G. C., Chao, A. C., Tsai, M. C., Sung, H. F. (2004). Decolorizing of lignin wastewater using photochemical UV/TiO₂ process. Chemosphere, 56(10)1011-1017.
26. Allen, N. S., Edge, M., Ortega, A., Sandoval, G., Liauw, C. M., Verran, J., Stratton, J., McIntyre, R. B. (2004). Degradation and stabilisation of polymers and coatings: nano versus pigmentary titania particles. Polym. Degrad. Stabil.. 85(3), 927-946.
27. Allen, N. S., Edge, M., Ortega, A., Liauw, C. M., Stratton, J., McIntyre, R. B. (2006). Factors affecting the interfacial adsorption of stabilizers on to titanium dioxide particles (flow microcalorimetry, modeling, oxidation and FTIR studies): Nano versus pigmentary grades. Dyes Pigments., 70(3), 192-203.
28. www.wiley-vch.de/books/biopoly/pdf_v03b/bpo13b10_{—}265_{—}274.pdf

1. A process of producing short chain carboxylic acids by controllably photocatalytically degrading lignin residues in aqueous solution comprising treating said lignin residues in the presence of a titanium dioxide nanocatalyst with ultraviolet radiation.

2. A process as claimed in claim 1 wherein said lignin residue is selected from the group consisting of phenol, syringol and guaiacol.

3. A process as claimed in claim 1 wherein said lignin residue is present in an aqueous waste stream.

4. A process as claimed in claim 3 wherein said waste stream is a recalcitrant efficient stream rich in lignin model compounds.

5. A process as claimed in claim 1 comprising an oxidising agent.

6. A process as claimed in claim 5 wherein said oxidising agent is selected from the group consisting of dissolved oxygen, hydrogen peroxide and ozone.

7. A process as claimed in claim 1 wherein said short chain carboxylic acid comprises a C₂—C₁₀ linear or branched chain alkyl or alkenyl group.

8. A process as claimed in claim 7 wherein said short claim carboxylic acid is selected from the group consisting of formic acid, acetic acid, succinic acid, maleic acid, fumaric acid and their derivatives.

9. A process as claimed in claim 1 wherein said titanium dioxide has a nanoparticle size of at least 10 nm.

10. A process as claimed in claim 1 wherein said titanium has a nanoparticle size selected to provide an activation energy of between 5-50 kilojoule per mol of said lignin residue.

11. A process as claimed in claim 9 wherein said aqueous solution has a pH of less than 7.

12. A process as claimed in claim 11 wherein said pH is selected from 1 to 3.

13. A process as claimed in claim 1 wherein said titanium dioxide nanocatalyst is immobilized on a support material.

14. A process as claimed in claim 1 selected from a batch process and a semi-continuous mode.

15. An aqueous solution comprising a short-chain fatty acid when produced by a process as claimed in claim 1.

16. A short-chain fatty acid when isolated from a solution as defined in claim 15.

17. A process as claimed in claim 4 wherein said lignin residue is selected from the group consisting of phenol, syringol and guaiacol.

18. A process as claimed in claim 17 comprising an oxidising agent.

19. A process as claimed in claim 18 wherein said oxidising agent is selected from the group consisting of dissolved oxygen, hydrogen peroxide and ozone.

20. A process as claimed in claim 19 wherein said short chain carboxylic acid comprises a C₂-C₁₀ linear or branched chain alkyl or alkenyl group.
